(19) United States
(12) Patent Application Publication
(10) Pub. No.: US 2016/0000990 A1
(43) Pub. Date: Jan. 7, 2016

(54) DIALYSIS MACHINE

(71) Applicant: B. BRAUN AVITUM AG, MELSUNGEN (DE)

(72) Inventor: KAI-UWE RITTER, REDNITZHEMBACH (DE)

(21) Appl. No.: 14/755,516

(22) Filed: Jun. 30, 2015

(30) Foreign Application Priority Data
Jul. 4, 2014 (DE) 10 2014 109 369.0

(51) Int. Cl. A61M 1/36

(52) U.S. Cl. CPC A61M 1/3663

(57) ABSTRACT

A dialysis machine which includes a preparation system comprising a main line for the dialysis liquid, several supply lines for respective concentrates opening at said main line is disclosed. At a point behind each orifice a respective mixing chamber is arranged and after the latter a respective conductivity probe is disposed. Downstream of the last conductivity probe, a shared pump is arranged. The negative pressure in the main line is adjusted via a pressure control valve which is arranged upstream of the first orifice in the main line. Furthermore, a proportional valve or a digital switching valve is disposed for metering the various concentrates in each supply line. The pressure control valve and the proportional valves or switching valves are controlled in electromagnetic fashion by a central electronic control unit.

### CROSS REFERENCE TO RELATED APPLICATIONS

This application claims priority to German application DE 10 2014 109 369.0 filed Jul. 4, 2014, the contents of such application being incorporated by reference herein.

### FIELD OF THE INVENTION

The invention relates to dialysis machines with which the blood of a patient can be cleaned if for instance his/her renal function is restricted or has ceased. To this end, the dialysis machine comprises a dialyzer in which there is a flow, on the one hand, of the patient's blood to be cleaned and on the other hand of a dialysis liquid, preferably according to the counter-current principle; in this process, certain dissolved substances (e.g. urea) from the blood migrate to the dialysis liquid.

The invention relates in particular to the preparation of the dialysis liquid before it is supplied to the dialyzer. Here, water preferably coming from a central osmosis-based unit is degassed first and then concentrates (e.g. bicarbonate and acidic concentrate) are added. The concentrates are made available in different forms, in particular in canisters from where the respective concentrate has to be drawn in via suction rods, or via a central concentrate supply system (ZKV) whose supply lines are under overpressure. The bicarbonate may also be provided in powder form in cartridges. Prior to the addition to the dialysis liquid, water is pressed through the cartridge in order to produce a saturated bicarbonate solution.

After the addition of each of the concentrates, the dialysis liquid is conveyed through a respective conductivity meter in order to detect the concentration of the respective concentrate in the dialysis liquid and control its (further) addition.

### BACKGROUND

For the purpose of adjusting the respective addition quantity of concentrates, the prior art knows volumetric pumps which are arranged in a respective concentrate supply line opening into a central main line, transporting the dialysis liquid, of the preparation system.

This is disadvantageous in that the pumps used today are expensive whereas their service life is limited due to wear and tear. Furthermore, there is the problem that the concentrates may crystallize and hence may result in a jamming of the pump. If bicarbonate cartridges are used, filling and emptying needs a long time. In the course of a disinfection, the pumps have to be run with maximum speed in order to rinse the supply line with maximum flow, resulting in increased wear and unpleasant noise pollution.

If rotary slide piston pumps are used, the crystals of the concentrate may cause leaks on the pump. Rotary slide piston pumps require additional rinse lines which are expensive in terms of device engineering and hence costly; what is more, they have to be disinfected. In case of using a central concentrate supply system, rotary slide piston pumps are prone to leaks, as their input side is constantly under pressure.

### DESCRIPTION OF THE RELATED ART

From DE 27 45 572, a preparation system for a dialysis liquid is known, in which a supply throttling valve is arranged in the concentrate supply line carrying a saline solution concentrate and opening into the main line for the dialysis liquid. The supply throttling valve is controlled via a regulator as a function of a conductivity meter determining the concentration of the saline solution in the dialysis liquid. As seen in the flow direction of the dialysis liquid, the main line is provided first with a main throttling valve, followed by an orifice of the concentrate supply line and then an adjustable pump which is controlled as a function of a pressure probe, arranged in the area of the orifice, of a pressure controller.

For the currently usual flow rates of the dialysis liquid in the range from 100 to 1000 ml/min, the solution according to DE 27 45 572 results in a very large change in the negative pressures at the orifice of the concentrate supply line. In this case, the negative pressure has to be designed such that the concentrate is reliably sucked in even with 100 ml/min. This is why elevated flow rates from 800 to 1000 ml/min result in very high negative pressures which give rise to noise pollution and a high pump output. In this context, outgassing of the dialysis liquid may occur at the orifice and in the pump, the latter resulting in cavitation damages. The adjustment or control is difficult, because the supply throttling valve (flow control valve) of the concentrate supply line is subject to a steep control gradient.

Such a preparation system for a dialysis liquid has the disadvantage that the pump generates a negative pressure in the main line downstream of the main throttling valve, said negative pressure being highly dependent on the flow. The pressure at the orifice of the concentrate supply line is to be controlled via the pump speed of the pump. This only works for a specific flow. The flow and the negative pressure cannot be separately adjusted with this preparation system.

### SUMMARY OF THE INVENTION

Compared to this, aspects of the invention are based on the object to provide a dialysis machine in which the afore-mentioned disadvantages are avoided.

According to one aspect of the invention, the claimed dialysis machine comprises a preparation system including a main line for a dialysis liquid, one or more supply lines for respective liquids (concentrates in the majority of cases) to be added to the dialysis liquid opening into said main line, and a (suction) pump is provided behind (downstream of) the orifices. With this arrangement, a negative pressure (with regard to the atmosphere) is produced in the main line, due to which the concentrates are drawn into the main line. According to aspects of the invention, an electromagnetically operated or preferably a pressure-operated as well as further preferred an adjustable controlled pressure control valve is arranged in the main line in front of (upstream of) the first orifice, and each supply line is provided with an electromagnetically controlled valve assembly (switching valve). This allows to always adjust the required minimum negative pressure in the main line even over a large range of different flow speeds of the dialysis liquid, improving the controllability of the concentrations. Further, the pump is not stressed more than necessary.

Despite of largely varying (adjustable) flow speeds, the dialysis machine according to aspects of the invention is able, for example, to adjust a negative pressure of −200 mbar in the main line as compared to atmospheric pressure, said negative pressure being sufficient to draw e.g. a concentrate into the main line from a canister with a supply line with a diameter of preferably 3 mm and through an altitude difference of up 70 cm. In the case of a bicarbonate cartridge, said main line can be quickly emptied and filled by completely opening the associated valve assembly. In the event of a disinfection of the dialysis machine according to aspects of the invention, the at least one valve assembly can be opened to its maximum degree (or permanently).

Particularly preferred is an electronic control unit which, on the one hand, is able to adjust preferably the pressure control valve and on the other hand is capable of adjusting the at least one valve assembly as a function of a conductivity measuring device associated to it. Said measuring device may be a conductivity probe which is preferably arranged in the main line.

So that the output signal of the conductivity measuring device is comparable even if the associated valve assembly dispenses the concentrate in unsteady or pulse-like manner, it is particularly preferred to arrange a mixing chamber between the associated orifice and the associated conductivity measuring device.

The at least one valve assembly may be a proportional valve. This allows to (continuously) adjust the supplied flow of the concentrate via its port size.

As an alternative, the at least one valve assembly may comprise a first digital (electromagnetically actuated) switching valve (2/2-switching valve). In that case, the supplied flow of the concentrate can be adjusted with different, in particular periodically recurring opening impulses (in amplitude-, pulse width- or frequency-modulated fashion).

With a further development of the dialysis machine according to aspects of the invention comprising the mixing chamber, the frequency and/or pulse width of the opening impulse of the associated switching valve is preferably selected such that several opening operations of the switching valve occur during filling the mixing chamber. This ensures that the mixing chamber is capable of mixing and preparing the dialysis liquid and the concentrate in the best possible way.

In a preferred configuration, a first supply line comprising a first switching valve and a second supply line comprising a second switching valve are connected to the main line, the two switching valves preferably having equal or different port sizes.

Here, bicarbonate from a canister may be supplied at the first supply line and an acidic concentrate from a further canister may be provided at the second supply line. In this case, the dialysis machine must be able to detect an accidental mix-up of canisters. In fact, if two bicarbonate canisters were to be connected by mistake, this could result in an alkalosis of the patient. This risk can be tackled if a valve in the route of the dialysis liquid preparation is closed at the beginning of a therapy and if only concentrates are drawn in without water, so that checking the conductivity allows to exclude any undesired liquids.

Due to the conductivity ratios, the opening period of the first switching valve has to be substantially longer than that of the second switching valve. This ratio as well as an admissible range of the opening periods can be checked by the electronic regulating/control unit.

If bicarbonate from a canister is supplied at the first supply line and an acidic concentrate from a central concentrate supply system is provided at the second supply line, then it is not necessary to monitor the central concentrate supply system. It is possible, however, to ensure a safety measure to the effect that an admissible range of the opening periods of the first switching valve is not exceeded.

If both the bicarbonate and the acidic concentrate are made available via the central concentrate supply system, a check is not required.

If the bicarbonate is provided from a cartridge and the acidic concentrate from a canister, the central concentrate supply system does not have to be monitored. It is possible, however, to ensure a safety measure to the effect that an admissible range of the opening periods of the second switching valve is not exceeded.

This allows to prevent a wrong metering, in particular of the bicarbonate, due to the mixing up of canisters.

The at least one valve assembly may comprise a further digital switching valve which is connected in parallel to the first switching valve. In this case, two different permanent or nominal cross-sectional areas of the valve assembly can be realized if the port sizes are equally large, and three port cross-sectional areas can be realized if the port sizes are different. This allows to select a large cross-sectional area, for instance in the case of drawing the concentrate out of a canister, and if the concentrate is delivered from the central concentrate supply system, its overpressure allows to select a smaller cross-sectional area.

In addition, it is also possible in this further development to adjust the supplied flow of the concentrate via successive opening impulses.

If the port sizes of the two switching valves of the respectively one valve assembly have a ratio of 1:2, the gradation possibilities of the valve assembly are optimized. It is also possible to provide more than two parallel switching valves for each valve assembly, whose port sizes are graduated preferably in the ratio 1:2:4:8:16 etc.

With the further development comprising only one switching valve per valve assembly and the further development comprising several switching valves per valve assembly, a metering of the concentrate can be effected in that the control unit transmits opening impulses of differing lengths at a constant frequency (pulse width modulation method), or opening impulses with the same length but with different frequencies (pulse frequency modulation method).

Having a mixing chamber volume of 100 ml, a metering with 3 to 5 opening operations per 100 ml would result in an advantageous operation. With an assumed service life of 20,000 h for a valve, there are 30 to 50 million switching cycles of the associated switching valve depending on the flow of the dialysis liquid. A higher number of opening operations is advantageous if valves are available which have an appropriate service life. A smaller number of opening operations increase the pulsation of the conductivity of the dialysis liquid, so that a sufficient smoothing of the conductivity is impeded. The opening frequency depends on the flow, and 4 opening operations/min are recommended with a flow of 100 ml/min, 20 opening operations/min with a flow of 500 ml/min and 32 opening operations/min with a flow of 800 ml/min.

In order to reach an especially high error safety of the dialysis machine according to aspects of the invention, a final conductivity measuring device, connected to a monitoring equipment, is arranged preferably in the main line between the last conductivity measuring device and the pump. The conductivity measuring device may be a conductivity probe.

In a preferred further development of the dialysis machine according to aspects of the invention, an electromagnetically controlled shutoff valve is arranged in the main line in front of (upstream of) the orifices of the feed lines. This allows to carry out a test in which a possibly wrong concentrate or a wrong canister is identified. To this end, the shutoff valve is closed so that the pump does not suck in water but only the (at least one) concentrate. A measurement of the conductivity by the (at least one) associated conductivity measuring device can be matched with (at least one) deposited value.

### BRIEF DESCRIPTION OF THE DRAWINGS

The invention is best understood from the following detailed description when read in connection with the accompanying drawings. Included in the drawings are the following figures:

FIG. 1 shows an exemplary embodiment of the dialysis machine according to aspects of the invention;

FIG. 2 shows schematic diagrams depicting different conductivities of a dialysis liquid of the dialysis machine according to FIG. 1;

FIG. 3 shows a diagram depicting a signal of a switching valve and a conductivity in front of an associated mixing chamber of the dialysis machine according to FIG. 1; and

FIG. 4 shows a diagram depicting a signal of a switching valve and a conductivity after an associated mixing chamber of the dialysis machine according to FIG. 1.

### DETAILED DESCRIPTION OF THE PREFERRED EMBODIMENTS

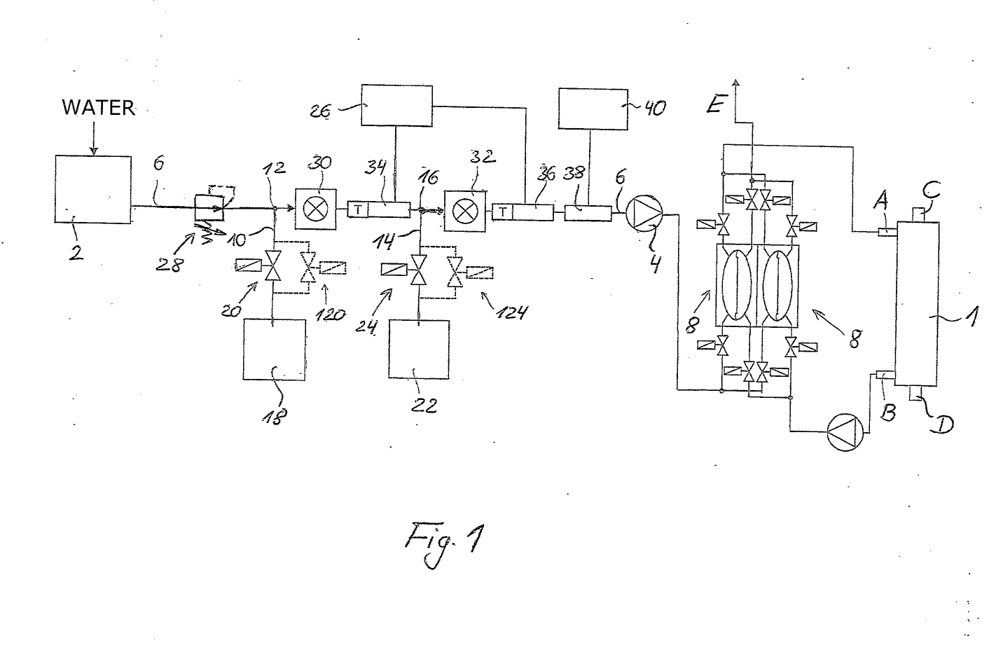

FIG. 1 shows a circuit diagram of an exemplary embodiment of a dialysis machine according to aspects of the invention. It comprises a dialyzer 1, and on the one hand there is a flow of a dialysis liquid through said dialyzer via ports A, B and, on the other hand, patient's blood to be cleaned flows through said dialyzer via ports C, D. The dialysis machine further comprises a preparation system for the dialysis liquid. A water supply system delivers water to a tank 2 in which a degassing process takes place. From there, the water or the dialysis liquid is drawn by a pump 4 through a main line 6 and then conveyed through a system including two balancing chambers 8. Each of the balancing chambers 8 has a connection to the main line 6 for freshly prepared dialysis liquid, a connection to the port A of the dialyzer 1, a connection to the port B of the dialyzers 1 and a connection to a drain E for used dialysis liquid. Each balancing chamber 8 is subdivided into two chamber portions by a membrane. The system comprising the two balancing chambers 8 ensures that the amount flowing to the dialyzer 1 is equal to the amount flowing off the dialyzer 1.

In the illustrated exemplary embodiment of the preparation system, a first concentrate of bicarbonate is supplied to the water via a first supply line 10 which opens into the main line 6 at a first orifice 12. At a point downstream thereof, a second supply line 14 opening into the main line 6 at a second orifice 16 supplies a second, acidic concentrate to the dialysis liquid.

A first digital switching valve 20 is arranged in the first supply line 10 which is immersed in a first canister 18, whereas a second digital switching valve 24 is arranged in the second supply line 14 which is immersed in a second canister 22. Both switching valves can be switched to an opening position with a respective electromagnetic actuator of an electronic control unit 26.

A pressure control valve 28 is arranged in the main line 6 between the first orifice 12 and the tank 2. In a preferred configuration, the pressure control valve 28 can also be electromagnetically controlled by the control unit 26. With this, the negative pressure generated by the pump 4 in the main line 6 is controlled in such a manner that there is a pressure at the two orifices 12, 16 which is required for drawing in the concentrate (e.g. −200 mbar).

Downstream of the orifices 12, 16, there are mixing chambers 30, 32 which are associated to one of the orifices in each case, and downstream of the mixing chambers, there are conductivity probes 34, 36 which are associated to one of the orifices in each case.

In operation of the dialysis machine according to aspects of the invention, the control unit 26 applies opening impulses according to the pulse width or pulse frequency modulation method to the respective electromagnetic actuator of the switching valves 20, 24. In doing so, the summed-up opening period of the respective switching valve 20, 24 is controlled as a function of a signal of the associated conductivity probe 34, 36, via which the concentration of the associated concentrate is determined.

Arranged between the second conductivity probe 36 and the pump 4 is a further conductivity probe 38 which transfers its signal to a final electronic monitoring equipment 40 for checking the conductivity of the readily prepared dialysis liquid.

In another exemplary embodiment of the dialysis machine according to aspects of the invention, a respective further switching valve 120, 124 is provided parallel to each switching valve 20, 24 (in FIG. 1 illustrated in broken lines). This allows to realize different nominal port sizes of the valve assembly formed in this manner, in particular if the two switching valves provided in parallel connection have different port sizes.

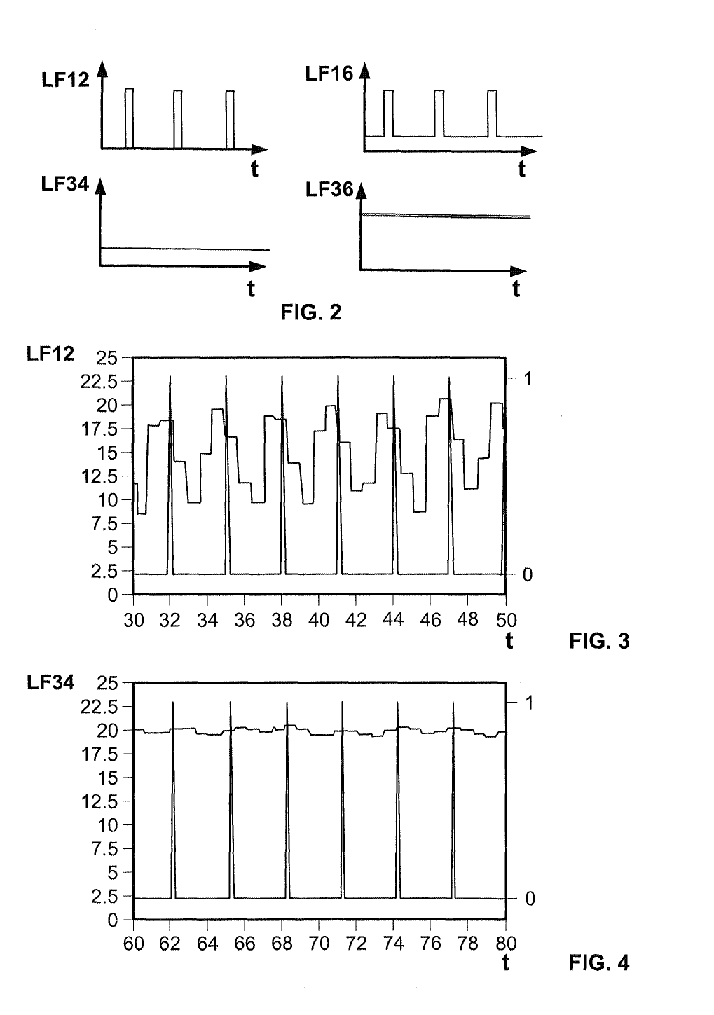

FIG. 2 schematically shows in four diagrams the conductivities of the dialysis liquid which are to be expected over the time t at four positions in the main line 6. To be more precise, the dialysis liquid shows the pulse-like conductivity LF12 behind the first orifice 12, i.e. after the bicarbonate concentrate has been added with opening impulses of the first switching valve 20. Downstream of the first mixing chamber 30, the first conductivity probe 34 determines the smoothed conductivity LF34. Downstream of the second orifice 16, i.e. after the acidic concentrate has been added with opening impulses of the second switching valve 24, the dialysis liquid shows the pulse-like conductivity LF16. Downstream of the second mixing chamber 32, the second conductivity probe 36 determines the smoothed conductivity LF36.

The temporally constant conductivity LF36 of the dialysis liquid is also present in a line between the balancing chamber 8 and the port A of the dialyzer 1.

FIG. 3 shows versus time t the periodical opening impulses of the first switching valve 20 for the bicarbonate concentrate and the measured, unsteady conductivity LF12 of the dialysis liquid downstream of the associated first orifice 12.

FIG. 4 shows versus time t the periodical opening impulses of the first switching valve 20 for the bicarbonate concentrate and the conductivity LF34 of the dialysis liquid measured by the first conductivity probe 34 at a position behind the first mixing chamber 30. The mixing of the bicarbonate concentrate with the water in the first mixing chamber 30 results in a smoothing of the course of the conductivity and hence can be used as a control variable for the regulator of the electronic control unit 26, said regulator adapting the metering of the bicarbonate concentration according to the pulse width or pulse frequency modulation method.

Departing from the first exemplary embodiment according to FIG. 1, a bicarbonate cartridge or a central concentrate supply system may be connected instead of the first canister 18. Said central concentrate supply system may also be connected instead of the second canister 22.

The disclosure relates to a dialysis machine which includes a preparation system comprising a main line for the dialysis liquid, several supply lines for respective concentrates opening at said main line. Downstream of each orifice, there is arranged a respective mixing chamber and after the latter a respective conductivity probe is disposed. Downstream of the last conductivity probe, a shared pump is arranged. The negative pressure in the main line is adjusted via a pressure control valve which is arranged upstream of the first orifice in the main line. Furthermore, a proportional valve or a digital switching valve is disposed for metering the different concentrates in each supply line. The pressure control valve and the proportional valves or switching valves are controlled in electromagnetic fashion by a central electronic control unit. In other words, there is provided an assembly for preparing a dialysis liquid which does without volumetric pumps, the latter being replaced by valves. With this assembly, controlling the volume or dosage is carried out with a control loop via measuring the conductivity. In this process, the hydraulic conditions in the system are adapted such that a relative negative pressure generated in this way is sufficient for the suction of individual liquids. In addition, a specific assembly as well as a particular chronological order of opening the valves improve the safety against mixing up the containers.

1-12. (canceled)

13. A dialysis machine comprising a preparation system for a dialysis liquid, the preparation system comprising:
a main line for the dialysis liquid to which a supply line for a liquid to be added to the dialysis liquid is connected via an orifice;
a pump in the main line downstream of the orifice;
a pressure control valve in the main line upstream of the orifice; and
an electromagnetically controlled valve assembly in the supply line.

14. The dialysis machine according to claim 13, further comprising:
an electronic control unit adapted to adjust at least the valve assembly as a function of an associated conductivity measuring device in the main line.

15. The dialysis machine according to claim 14, further comprising a mixing chamber associated with the conductivity measuring device and arranged between the orifice and the associated conductivity measuring device.

16. The dialysis machine according to claim 13, wherein the valve assembly is a proportional valve.

17. The dialysis machine according to claim 13, wherein the valve assembly comprises a switching valve.

18. The dialysis machine according to claim 17, wherein the switching valve is a 2/2-switching valve type.

19. The dialysis machine according to claim 17, further comprising:
a first supply line comprising a first switching valve; and
a second supply line comprising a second switching valve, the first and the second switching valves having the same or different port sizes.

20. The dialysis machine according to claim 17, wherein the valve assembly comprises a further switching valve arranged parallel to the switching valve.

21. The dialysis machine according to claim 20, wherein each of the switching valve and the further switching valve is a 2/2-switching valve type.

22. The dialysis machine according to claim 20, wherein the port sizes of the switching valve and the further switching valve of a respective valve assembly have a ratio of 1:2.

23. The dialysis machine according to claim 17, wherein opening impulses with differing lengths can be transferred to at least one of the switching valves.

24. The dialysis machine according to claim 23, wherein the opening impulses have a constant frequency.

25. The dialysis machine according to claim 17, wherein opening impulses with the same length but with differing frequencies can be transferred to at least one of the switching valves.

26. The dialysis machine according to claim 15, wherein a further conductivity measuring device whose signal can be transferred to monitoring equipment is arranged in the main line between the conductivity measuring device and the pump.

27. The dialysis machine according to claim 13, wherein an electromagnetically controlled shutoff valve is arranged in the main line upstream of the orifice.

28. The dialysis machine according to claim 13, wherein the pressure control valve is pressure-operated and adjustable.
